(19) United States
(12) Patent Application Publication
(10) Pub. No.: US 2021/0100491 A1
(43) Pub. Date: Apr. 8, 2021

(54) SYSTEM FOR USE IN IMPROVING COGNITIVE FUNCTION

(71) Applicant: BrainBerry Ltd, Leicestershire (GB)

(72) Inventor: Kartheka Bojan, Leicestershire (GB)

(21) Appl. No.: 17/044,368

(22) PCT Filed: Apr. 4, 2019
(86) PCT No.: PCT/GB2019/050987

§ 371 Date: Oct. 1, 2020

(30) Foreign Application Priority Data
Apr. 4, 2018 (GB) 1805555.8

(51) Int. Cl. A61B 5/378; A61B 5/372; A61B 5/00

(52) U.S. Cl. CPC A61B 5/378; A61B 5/372; A61B 5/744; A61B 5/0006; A61B 5/0022; A61B 5/4088

(57) ABSTRACT

A system comprising an EEG headcap, personal computer and cloud sewer system is configured to deliver a customised programme of activities to a user, based on an analysis of the EEG output from the user. The system is indicated in the treatment of mild cognitive disorder and early-stage dementia.

The present invention relates to a system which monitors and analyses the brain activity of a user, and which provides a customised programme of activity to the user.

The system of the present invention is particularly suitable for use by patients presenting with cognitive impairment or with symptoms that may be attributable to the onset of dementia.

The term ‘dementia’ refers to a group of symptoms associated with an ongoing decline of brain functioning. Alzheimer's disease and vascular dementia make up the vast majority of cases of dementia, with Alzheimer's disease (AD, or Alzheimer's) considered to be the cause of 60-70% of cases of dementia. The most common early symptom is difficulty in remembering recent events (short-term memory loss). As the disease advances, symptoms may include problems with language, disorientation (including easily getting lost), mood swings, loss of motivation, not managing self-care, and behavioural issues. As a person's condition declines, they often withdraw from family and society. Gradually, bodily functions are lost, ultimately leading to death. Although the speed of progression can vary, the average life expectancy following diagnosis is three to nine years.

It is thought that mental and physical exercise and avoiding obesity may decrease the risk of AD. There are no medications or supplements that decrease risk. No treatments stop or reverse its progression, though some may temporarily improve symptoms. For example, medicaments comprising cholinesterase inhibitors (Donepezil, Rivastigmine and Galantamine) may delay worsening of memory, thinking, language and thought processes, by supporting the communication between the nerve cells in the brain through stimulating the production of acetylcholine. Alongside one of the above medications, Memantime, an NMDA receptor antagonist, may be prescribed. This medication blocks the effects of excess glutamate in the brain. Memantine may assist memory, reasoning, language and attention. Exercise programmes are beneficial with respect to activities of daily living and can potentially improve outcomes. Treatment of behavioural problems or psychosis due to dementia with antipsychotics is common but not usually recommended due to there often being little benefit and an increased risk of early death.

The cause of Alzheimer's disease is poorly understood. Genetic factors may account for a significant number of cases. Other risk factors include a history of head injuries, depression, and hypertension. The disease process is associated with plaques and tangles in the brain.

A probable diagnosis is based on the history of the illness and cognitive testing, with medical imaging and blood tests used to rule out other possible causes. Initial symptoms are often mistaken for normal ageing. Examination of brain tissue is needed for a definite diagnosis.

The presence of characteristic neurological and neuropsychological features and the absence of alternative conditions may be supportive of a diagnosis of Alzheimer's Disease. Advanced medical imaging with computed tomography (CT) or magnetic resonance imaging (MRI), and with single-photon emission computed tomography (SPECT) or positron emission tomography (PET) can be used to help exclude other cerebral pathologies or subtypes of dementia. Moreover, it may predict conversion from prodromal stages (Mild Cognitive Impairment) to Alzheimer's disease. Mild Cognitive Impairment (MCI) refers to a transitional stage between normal cognition and dementia, wherein the decline in cognitive function (which is mostly related to memory) does not interfere with a person's normal daily activities. Patients diagnosed with MCI are three times more likely to develop dementia, compared with people with unimpaired cognition.

Assessment of intellectual functioning including memory testing can further characterise the state of the disease. Medical organisations have created diagnostic criteria to ease and standardise the diagnostic process for practising physicians. A diagnosis can be confirmed with very high accuracy post-mortem when brain material is available and can be examined histologically.

The present invention is concerned with AD and with other types of dementia such as vascular dementia, and with cognitive impairment.

Quantitative electroencephalography (EEG) may be used as a tool in the diagnosis of dementia. Al-Qazzaz et. al., in ‘Role of EEG as Biomarker in the Early Detection and Classification of Dementia’ The Scientific World Journal, Volume 2014, Article ID 906038, http://dx.doi.org/10.1155/2015/906038, reviews the role of EEG as a biomarker based on signal processing to detect dementia in early stages and classify its severity.

Various applications of EEG are known in the art.

US 2014/0350431 relates to a headgear with sensors tor electrophysiology measurement and training. The headgear comprises a helmet with electrodes which are electrically connected to an electrical stimulation or measuring device. EEG and ERP signals may be measured while providing sensory stimulus to a person, allowing determination of abnormalities of electrical activity in the brain, for example.

US 2016/235351 discloses a computer implemented method for obtaining an electrical signal representative of the brain activity of a particular individual; processing the signal with respect to a pre-determined predictor from a library of predictors containing a plurality of pre-determined predictors; and outputting a visual indication of a personalised mental state or neurological condition of the individual.

US 2011/224571 relates to cortical plasticity in the motor system induced by theta burst stimulation (TBS) as a biomarker of abnormal neuroplasticity in conditions such as Autism Spectrum Disorders, schizophrenia, Alzheimer's disease and dementia, and discloses that a baseline response measured before applying TBS may be compared with a subsequent response, post-TBS application, and an assessment of cortical plasticity impairment made.

The brain is divided into a number of brain regions, including the occipital, temporal, parietal and frontal lobes. The occipital lobe is involved with the brain's ability to recognise objects, colours and visualisation. The temporal lobes are concerned with interpreting, short-term memory and processing auditory stimuli. The parietal lobes are connected with the processing of nerve impulses related to the senses, such as touch, pain, taste, pressure, and temperature, and they also have language functions. The frontal lobe is concerned with long-term storage memory, emotions, reasoning, planning, movement, and parts of speech; it is also involved in purposeful acts such as creativity, judgment, and problem solving, and planning.

Brain waves may be categorised into their different bandwidths (slow, moderate and fast), with brainwave speeds measured in Hertz (cycles per second):

Delta brainwaves (0.5 to 3 Hz) are slow, loud brainwaves which are generated in deepest meditation and dreamless sleep. Delta waves suspend external awareness and are the source of empathy. Healing and regeneration are stimulated in this state.

Theta brainwaves (3 to 8 Hz) occur most often in sleep but are also dominant in deep meditation. Theta is the gateway to learning, memory, and intuition.

Alpha brainwaves (8 to 12 Hz) are dominant during quietly flowing thoughts, and in some meditative states, and aid overall mental coordination, calmness, alertness, mind/body integration and learning.

Beta brainwaves (12 to 38 Hz) dominate the normal waking state of consciousness when attention is directed towards cognitive tasks and the outside world. Beta is a ‘fast’ activity, present when we are alert, attentive, engaged in problem solving, judgment, decision making, or focused mental activity. Beta brainwaves are further divided into three bands; Beta-1 (12-15Hz), Beta-2 (15-22Hz), and Beta-3 (22-38Hz).

Gamma brainwaves (38 to 42 Hz) are the fastest of brain waves and relate to simultaneous processing of information from different brain areas.

The present invention relates to a system for monitoring and improving cognitive function in a user, for example in a patient presenting with symptoms of early dementia. The system comprises an EEG headset; a personal computer and a cloud server system. The EEG headset monitors and records the brain activity of the patient under a specific condition and transmits information to a cloud server system which is pre-programmed with reference relating to the brain activity of healthy individuals, i.e., people having no early onset symptoms of dementia, under the same specific condition. Algorithms in the cloud server compare the brain activity of the patient under the specific condition with the brain activity of the healthy individuals under the same specific condition and identify points of difference, for example in activation of particular regions of the brain and/or in the type or properties of brain waves activated; and direct customised activities, for improving cognitive function, to the patient.

The system is designed for personal use by a patient in his or her own home and may for example be portable and wireless. The terms ‘individual’, ‘user’, and ‘patient’ are used interchangeably herein.

In one aspect, the present invention provides a system for monitoring and/or improving cognitive function in a user, said system comprising:
an EEG headset comprising electrodes positioned in areas corresponding to a user's brain lobes, a recorder, and a transmitter;
a personal computer configured with interactive activities; and
a cloud server system comprising a private cloud platform;

characterised in that:
the private cloud platform comprises an area in which photographs and images personal to the user may be uploaded;
the private cloud platform is configured with reference EEG profiles of healthy individuals; and
the private cloud platform is configured with algorithms for
(i) analysing EEG output of the user engaged with a specific interactive activity; and
(ii) instructing the personal computer in the presentation of specific interactive activities to the user.

The electrodes of the EEG headset detect the brain activity of a user, to give an activity profile which may be stored temporarily in the recorder and transferred via the transmitter to the private cloud platform in the server. Detection of the brain activity may be referred to as measurement of brain activity. The information which is transmitted to the private cloud platform may be referred to as ‘EEG output’, or ‘output’.

‘EEG output’ includes the identification of the brain area(s) activated with a particular activity as well as the brain wave(s) activated with a particular activity.

‘Analysing EEG output’ involves the comparison of specific properties of the brain activity of the user, in relation to a specific interactive activity, with the reference EEG profile of healthy individuals in relation to the same specific interactive activity and reaching a decision in respect of the specific interactive activity that should be delivered to the user via the personal computer.

Thus, the algorithms of the present invention analyse the brain activity (output) of a user engaged in interactive activity modules provided through the personal computer, including the specific brain regions activated during engagement with particular interactive activity; the types of brain waves activated; and comparison of the output with the appropriate reference brain profiles stored within the private cloud platform. The algorithm decision instructs presentation to the user of particular activities, with the aim of driving a cognitive improvement. No user choice is involved.

Reference profiles represent an average pattern of the brain wave activity of healthy individuals and may be gained through capturing brain wave activity of healthy individuals while engaged in the interactive activity modules. They are pre-programmed into the system of the present invention and are specific for each available interactive activity.

The EEG headset (or headcap) is a wearable headset and may take any suitable form. Electrodes may be referred to as ‘sensors’, The EEG headcap may also be referred to as a ‘recorder’.

The personal computer may be a tablet device and references herein to ‘tablet’ refer to a tablet device or other personal computer. The personal computer may also be referred to as an ‘activator’, as it stimulates the brain when being used.

The cloud server and private cloud platform with the algorithms may be referred to a ‘processor’.

As described above and in accordance with the present invention, a private cloud platform is configured with an area in which photographs and images personal to the user may be uploaded. Uploaded photographs and images are used in personalised activities, as are described in more detail below. Preferably, a patient's carer or other person will upload photos and images onto the system of the present invention before activities are presented.

Activities, or ‘games’, in accordance with the present invention are arranged in a number of different activity ‘modules’. These may include:

‘Reminiscence’

‘Visual Stimulation’

‘Meditation’

‘Feedback’

‘Spatial’

‘Language’

‘Tasks’ and

‘Decision Making’.

The above list is not to be considered as limiting, and other activity modules may be employed in the system of the present invention.

The Reminiscence module may comprise activities in which the user is presented with an item, such as a photograph, personal to him or herself, together with questions related to said personal items.

The Visual Stimulation module may comprise activities in which the user is presented with various colours and shapes, together with puzzles related to said colours and shapes.

The Meditation module may comprise focus, breathing, meditation and hypnotic meditation sessions.

The Feedback module may comprise a therapy session in which the user is asked questions about his or her current personal circumstances.

The Spatial module may comprise perceptual and visual games that set the user tasks such as map navigation and finding routes around familiar places such as the user's own home or neighbourhood.

The Language module may comprise language and comprehension tests.

The Tasks module may involve giving the user simple tasks to carry out, for example to make a cup of tea or prepare toast.

The Decision Making module may comprise games in which the user is asked to make decisions, including decisions corresponding to real-life situations.

Activities within each interactive activity module may be referred to as activities, or games. Games may be provided in a range of game levels, of varying (e.g. increasing) levels of difficulty.

During participation in an activity, electrodes in the EEG headcap monitor the brain activity of a user. Said monitoring may be carried out in repeated sessions over a fixed time period. For example, monitoring of brain activity may be carried out in daily sessions of engagement in a particular activity, for example over a period of seven days, during which time a given activity is continued without change. Algorithms in the cloud server analyse the user's brain activity over the fixed period with reference to stored reference profiles of the brain activity of healthy individuals and identify differences between the user's response and that of a healthy individual. At the end of the fixed period, the algorithms direct the presentation of specific activities to the user (via the personal computer), designed to improve the cognitive function of the user.

Participation in activity modules in accordance with the present invention has been demonstrated to upregulate alpha and beta activity, and to activate frontal, parietal and occipital lobes of users. Depending on the particular activity module and game being played, other brainwaves and brain regions may also be activated. The findings indicate use in the prevention and treatment of MCI and early dementia.

Thus, the system of the present invention is particularly designed for use by a person presenting with MCI and/or symptoms of early dementia.

Activity modules in accordance with the present invention are described in more detail below.

Reminiscence

The Reminiscence activity module is a personalised module which provides puzzles and games using tangible prompts such as photographs (for example, family photos), household items (for example, personal items) and other familiar items from a users's past, such as music and sound recordings specific to the user's history. The use of personal photographs and items in a game provokes the user's thinking process.

Games included in the Reminiscence activity module include the following:
Family Tree: In this game, the user is presented with an image of a family tree, labelled with titles of family members, such as ‘cousin’, ‘sister’, etc., and a series of photographs corresponding to said family members. The Game involves the user moving the photos provided to one of the labelled parts of the family tree.
Know Who: In this game, the user is presented with a photograph of a person familiar to them and is asked to enter that person's name.
Event: In this game, the user is asked to match details of, for example, a family event, with a photo, year, or name associated with that event.
Jigsaw Puzzle: In this game, the user is given a complete picture alongside a number of jigsaw puzzle pieces which together make up the picture; the user is asked to correctly place the pieces in an outline puzzle, in order to make up the picture.

Visual Stimulation

In this partly personalised activity module, the user is exposed to complex patterns of colour and geometry, in both 2-D and 3-D models, which he or she is asked to describe. This module may help in studying the cause of visuoperceptual symptoms, such as hallucinogenic phobias, as experienced by some dementia sufferers. Information obtained in accordance with the present invention may enable the reduction or prevention of such symptoms. In more detail, the system of the present invention can analyse a subject's response to the images presented during Visual Stimulation and potentially identify any behavioural changes that occur; enhancement of certain brain wave patterns, effected by stimulation in accordance with the present invention, may result in an avoidance of phobic and hallucinogenic reactions.

Games included in the Visual Stimulation module include:
Match Shapes and Colours: In this game, the user is presented with a set of outline shapes on one side of the tablet screen, and with circles of colour on another side of the screen. The user is then given a combination of a colour and a shape, for example. ‘orange heart’, ‘white octagon’, etc., and is asked to drag a colour onto a shape to make the requested combination.
Identify Blinking Patterns—In this game, the user is presented with abstract blinking patterns (for example, a pattern of dots); the patterns blink in fractions of seconds and the user is asked to identify the pattern.
Identify Blinking Colours—In this game, abstract colours blink in fractions of seconds and then the User is asked to identify the colours.
The above ‘blinking’ games are focused on increasing the concentration of the user and thereby increasing the Beta-1 activity in the user.
Identify Colours of Personal Objects—The user is asked to identify the colours of the personal pictures uploaded.

Meditation

In this general activity module, focus, breathing, meditation and hypnotic meditation sessions are delivered to the user.

Activities included in the Meditation module include:
Focus Point: this requires the user to focus on an image, for example a dot, and to reconstruct the image with eyes closed. A person with MCI or early-stage dementia may find difficulty in concentrating, and this session is aimed at improving concentration and lessening a wandering mindset.
Breathing: this session guides the user through a series of breathing exercises. A person with MCI or early-stage dementia may find it difficult to relax and feel calm, and thus this session aims to help a patient achieve a relaxed and calm state of mind.
Music: this session delivers to the user calming music, again to promote relaxation.

Positive Hypnosis—this delivers a hypnotic session to the user for positive experience and self-confidence.

Feedback

This is a personalised activity module to stimulate different functions of the brain, the user is presented with a series of questions about his or her self; for example, questions about his or her age and physical appearance.

This Activity module comprises a therapy session in which a set of psychological questions are posed to the patient. This module may raise an early stage dementia patient's awareness of his or her physical reality and current psychology. It is common for dementia patients to recall older memories, while more recent memories are easily depleted. For example, a 60-year-old dementia sufferer may believe that he/she is in his/her 40s and may therefore not recognise, or become frightened or confused by, an image of himself/herself. Feedback therapy in accordance with the present invention aims to create a feedback loop to the brain about the patient's present circumstances. In more detail, the software included in the present system records a video session in which the subject verbally answers questions posed by the system. The session is recorded and automatically replayed to the patient, for example for 3-5 times, and is configured in the software settings. This aims to register a new memory in the brain of the patient, to register information about himself/herself and his/her age and appearance. The feedback system will also monitor the regions activated during this session and keep track of the brainwave patterns observed and try to replicate those patterns during the therapies being played. This is an attempt to encounter new memory registration on the brain.

As mentioned above, games within the activity modules of the present invention may be provided at varying levels, referred to herein as ‘game levels’. Game levels may be graded for example from ‘very easy’ to ‘hard’, for example, with intermediate levels including ‘medium’ and ‘hard’. Factors determining the game level include: time allowed for a particular game, with the longest allowed time corresponding to the easiest, e.g., ‘very easy’ game level, and the shortest time allowed corresponding to the hardest, e.g., ‘hard’ game level; number of repeat attempts within a game in order to achieve a specific result, with the highest number of attempts allowed corresponding to the easiest, e.g., ‘very easy’ game level, and the lowest number of attempts allowed corresponding to the hardest, e.g., ‘hard’ game level; and where for example multiple choice is involved, the number of options to choose from, with the lowest number of choices presented corresponding to the easiest, e.g., ‘very easy’ game level, and the highest number of choices presented corresponding to the hardest, e.g., ‘hard’ game level.

It is envisaged that activities may employ avatars in personalised 3D games based on people and places familiar to the user.

Properties of brain waves monitored in accordance with the present invention may include one or more of the frequency, amplitude and morphology of the brain waves, with specific reference to the brain regions activated by each particular game.

As stated above, algorithms in the cloud server analyse the user's brain activity over a fixed period with reference to stored reference profiles of the brain activity of healthy individuals and identify differences between the user's response and that of a healthy individual.

Reference profiles from healthy individuals indicate a range of “acceptable” values for the brain wave properties in response to a specific activity, and permit assessment of the user's response.

Properties are typically monitored and analysed over an extended time period. Typically, monitoring and analysis may be carried out over an initial period of up to 4 weeks, for example 4 weeks, or 2-3 weeks, i.e., any number of days from 14 days to 21 days, During this time, an activity module is undertaken regularly, for example daily, or over several days each week.

In one embodiment, in use, the system according to the first aspect of the present invention compares the brain activity of a person presenting with one or more symptoms of early dementia with an average pattern of brain activity gained from the monitoring of a number of people with no early onset symptoms of dementia (the reference pattern or profile) for each game within an activity module. Thus, the profiles are individualised to specific games. Areas of difference between the brain activity observed in the person presenting with one or more early onset symptoms of dementia and the reference pattern, may be supportive of a diagnosis of dementia and may indicate a treatment pathway for the disease. For example, where areas of difference exist between the brain wave activity captured in the person presenting with one or more early onset symptoms of dementia and the reference pattern, the activity modules described herein may serve to restore or enhance brain wave activity as appropriate, so as to reduce or remove the differences between the captured and reference patterns. Thereby, the early onset symptoms leading to dementia may be alleviated and the system of the present invention can be used as an early intervention system.

A user of the system of the present invention does not have any choice in selecting activity modules or games since the algorithms within the system will analyse the user's brain activity and instruct the delivery of an activity module according to the requirement of the brain activity demanded as per the healthy brainwave profile collected for each game within each activity module.

Analysis of the frequency of a user's brain waves may be carried out while the user is engaged in a game within a given activity module. A reference brain wave frequency (f_{R}) for the given activity is stored within the system of the present invention. In one specific example, the acceptable range, or ‘condition’ for the property of frequency may be plus or minus 2 Hz of the stored reference for frequency. Thus, if the analysed frequency (f_{M}) is within 2 Hz of the stored reference frequency, it can be said to meet, or pass the condition. If the condition is not met, the system algorithms look for other games within the activity module to pass the condition of f_{M} within 2 Hz of the stored reference frequency

As stated above, monitoring and analysis is carried out over an extended time period. If at the end of an initial period there is identity between the analysed frequency f_{M} and the reference frequency f_{R}, i.e., if f_{M}−f_{R}=0, the activity module is discontinued. The activity module is also discontinued at this point if the analysed frequency is less than 8 Hz or more than 20 Hz, i.e., if 8 Hz≤f_{M}≤20 Hz. A frequency of less than 8 Hz may indicate boredom in the user, which may lead to a loss of interest in the system. Otherwise, the activity module is continued, and different games in the activity module are played.

Different Activity Modules may run concurrently over the time period concerned.

Over the initial period, a game is continued at the same game level. If the condition is not met within the four-week period, the game is discontinued. In this case, it can be said that the game is not effective for the user. If the condition is met, the game is continued, and an ‘Improvement Effect’ (I) determined. The Improvement Effect relates to the difference between f_{M} and f_{R}. The value of the difference is designated as ‘x’, with a positive or negative value for x indicating a difference between f_{M} and f_{R}. If I=±x within the initial period, a game will be continued at the same game Level; if I=0, the game will be continued and the game level increased.

Beyond the initial period, in a first following period, monitoring and analysis of brain activity continues. If, in this period, I=0 or I=±x, a game will be continued and the game level increased. After eight weeks, monitoring and analysis of brain activity may continue. If I=±x, a game will be continued and the game level increased; and if I=0, a different game is delivered.

In addition to frequency, analysis of the amplitude and morphology of the monitored brain waves can determine an appropriate game level based.

A stored a reference brain wave amplitude pattern for a given activity provides a rectangular window pattern of amplitude; each window may represent a time period of, for example, 0.99 s with a gap of 0.01 s between adjacent windows, and the maximum amplitude within each window will provide the reference values against which the amplitude of a subject's captured brain waves is compared. In one specific example, the acceptable range, or ‘condition’ for the property of amplitude is that 90% of the windows within a given time period, for example a time period of 600 seconds (therefore, 540 of 600 windows, for example), have a maximum amplitude of within 30% of the reference amplitude. Thus, if the analysed amplitude is within 30% of the stored reference amplitude for at least 90% of the captured time windows, it can be said to meet, or pass the condition.

A stored reference brain wave morphology pattern is stored for a given activity. The maximum brain wave amplitude observed within each of a series of rectangular windows (for example of 10 rectangular windows within a 0.99 s width, with a 0.001 s gap width between windows) constructs a shape of the brain wave, each maximum value giving a reference value for the rectangular window against which the amplitude of a subject's captured brain waves is compared. In one specific example, the acceptable range, or ‘condition’ for the morphology is that 70% of the windows within a given time period, for example a time period of 1 second (therefore, 7 of 10 windows, for example), have a maximum amplitude of within 40% of the reference amplitude.

During the initial period, patient-specific artefacts are monitored and assessed. These may include, for example, the patient's eye movements, blinking patterns, and head movements.

These are stored in the private cloud platform and configured in the algorithm alongside the reference EEG data. In this way, the effects of background ‘noise’ may be eliminated in the analysis of brain activity.

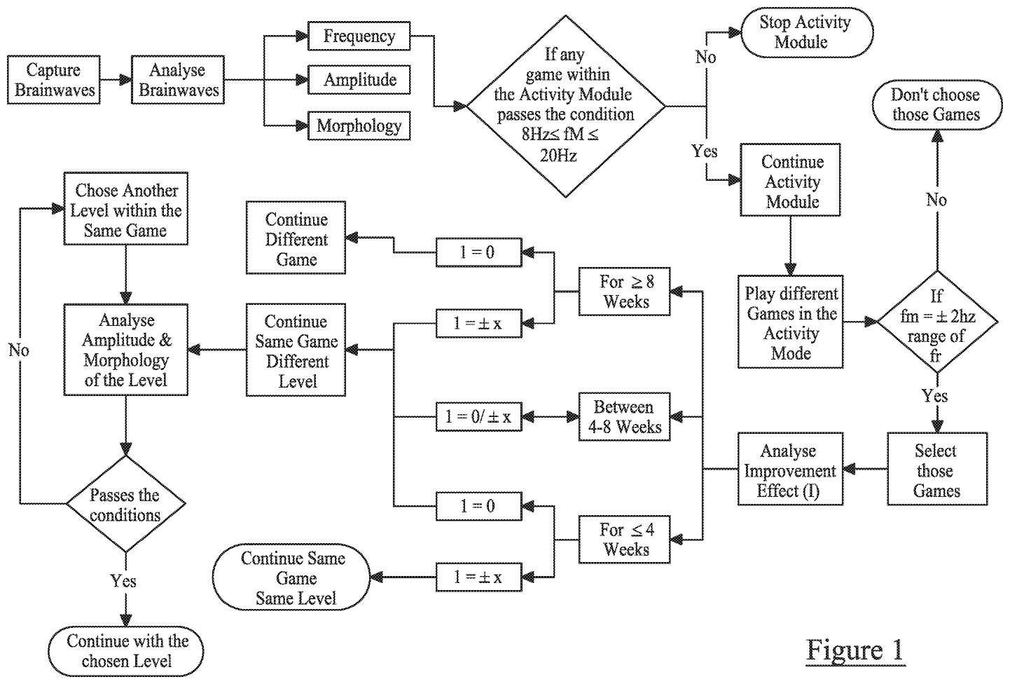

FIG. 1 herein illustrates an algorithm process of the present invention, as described above.

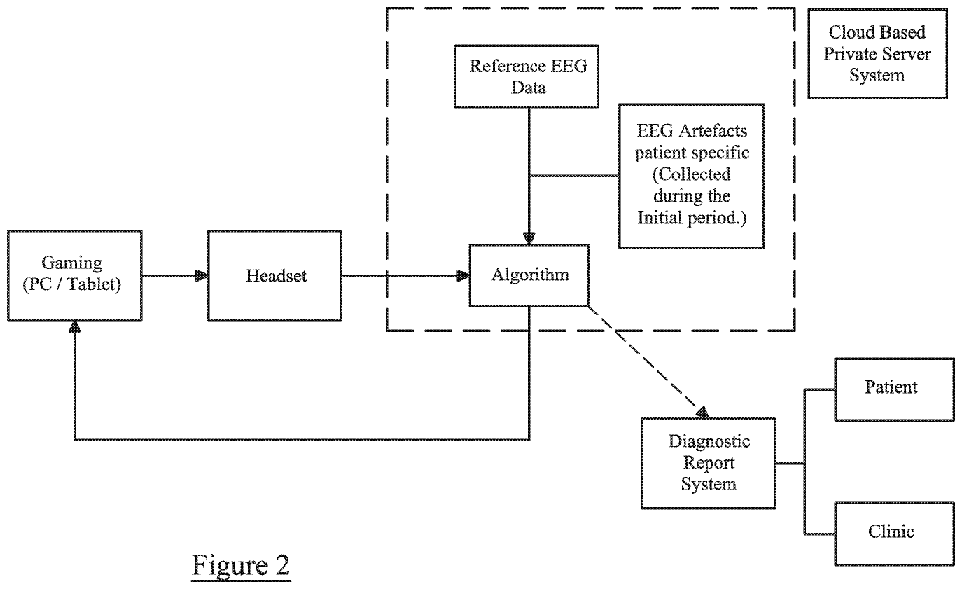

FIG. 2 gives an overview of the system of the present invention.

As can be seen from FIG. 2, the system of the present invention may include a diagnostic report system, in which a report of brain activity is generated for assessment by a clinician. The report will generally comprise the results of brain activity measured over an extended period of time, such as over a period of about 6 months.

Thus, in a second aspect, the present invention provides a method of supporting a diagnosis of MCI or early-stage dementia in a patient through use of the system of the present invention for an extended period and generation of a diagnostic report.

In further aspects, the present invention provides methods for monitoring the brain activity of a patient, improving cognition in a patient, and treating MCI and/or or early-stage dementia in a patient, through use by the patient of the system of the present invention.

The system of the present invention is designed for example for long-term use with repeated analysis of brain activity and consequential changes in games and game levels presented to the user.

The following study example indicates utility of the system of the present invention in the treatment of MCI and early-stage dementia.

### EXAMPLE

### Use of a Software-Based “Gamified Platform” for Cognitive Improvement

The study evaluated whether a software-based “gamified platform” can support cognitive improvement in healthy subjects. The study included 20 healthy volunteers (‘participants’) using a system in accordance with the present invention and was carried out over a period of one week. The brain activity of each participant was measured with EEG whilst using/playing activity modules as detailed below. Brain activity was analysed on Day 1 (first time using the software) and Day 7 (Software used for 7 days).

Reminiscence

Participants were delivered four games, each using pictures of family members and significant family events. The games were ‘Family Tree’, Know who', Jigsaw' and ‘Events’, all as described above.

The study found that participation in this activity module activated beta-1 activity in the brain, predominantly in the frontal and parietal brain regions. Parietal lobe activation was observed during episodic retrieval tasks, such as in the Event game. The most dominant frequency was beta-1, thus indicating the state of consciousness when attention is directed towards cognitive tasks and the outside world.

Significant upregulation in beta-i activity was seen with all games, especially in the dominant frontal lobe. The parietal lobe was activated in a 25% of participants during the Events Game, indicating the retrieval of old memories (this was confirmed by the participants). Significantly, these 25% of participants were older than 40 years of age, suggesting that this game can be satisfactorily used in the retrieval of memory in older adults.

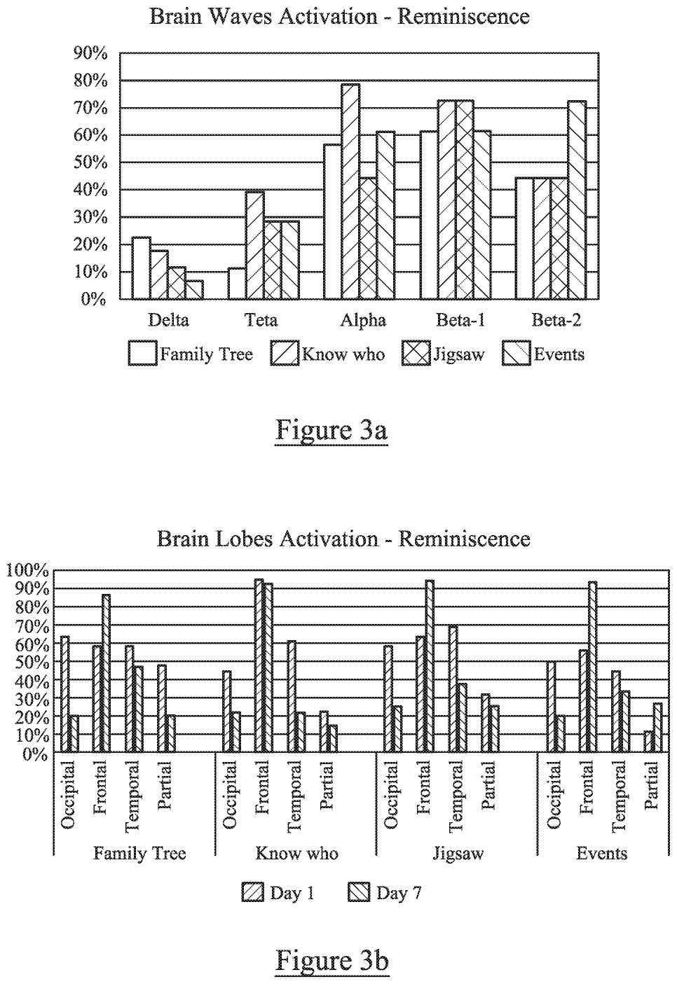

The results are illustrated in Table 1 below and in FIGS. 3a and 3b.

TABLE 1
Reminiscence-Percentage of Subjects showing Brainwave Activity

| | Family Tree | Know Who | Jigsaw | Events |
|---|---|---|---|---|
| Delta | 22% | 17% | 11% | 6% |
| Theta | 11% | 39% | 28% | 28% |
| Alpha | 56% | 78% | 44% | 61% |
| Beta-1 | 61% | 72% | 72% | 61% |
| Beta-2 | 44% | 44% | 44% | 72% |

A paired t-test confirmed the domination of Beta-1 brainwaves in all the four games or entire Reminiscence module.

TABLE 2
Paired t-test calculation (Reminiscence)

| | Paired T-Test Calculation | | | | |
|---|---|---|---|---|---|
| | | | Statistically | | |
| | | | Significant | Size Effect (% of | |
| Modules | Brainwaves | p-value | (p > 0.05) | participants) | |
| Family | Beta-1 | 0.002 | Yes | 85% | Large |
| Tree | | | | | |
| Know | Alpha | 0.07 | No | 46% | Medium |
| Who | Beta-1 | 0.03 | Yes | 55% | Medium |
| Jigsaw | Beta-1 | 0.03 | Yes | 65% | Medium |
| Events | Alpha | 0.02 | Yes | 62% | Medium |
| | Beta-1 | 0.03 | Yes | 54% | Medium |
| | Beta-2 | 0.11 | No | 40% | Low |

Visual Stimulation:

Participants were delivered a Visual Stimulation activity module as described above Brain wave frequency shifts of at least 0.5 Hz were observed.

The study found that 7 days participation in the Visual Stimulation activity module resulted in enhancement of mostly alpha and beta waves in the participants' frontal and partial lobes. The activation of the temporal lobe was significantly reduced, and the results indicate that new memories were encoded in this brain region and then consolidated in the frontal lobes for long-term storage. On Day 7, frontal lobe activation was dominant in more than 90% of participants, confirming its importance in storing memories. Nearly 50% of participants also showed an activation in their parietal lobes, confirming parietal lobe activation during episodic retrieval tasks.

The present results indicate that parietal lobe activation occurs in tasks that involve remembering colours (especially colours difficult to identify due to different shades of colours). The activation of the parietal lobe indicates intact decision making (retrieval of memory for colours) by most participants.

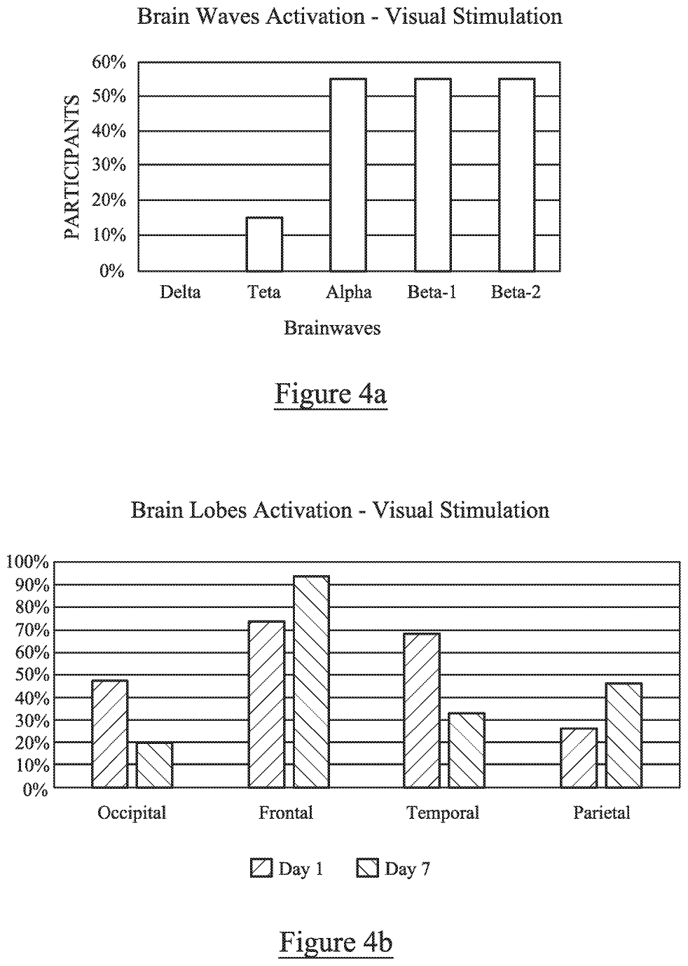

The results are illustrated in Table 3 below and in FIGS. 4a and 4b.

TABLE 3
Visual Stimulation-Percentage of subjects showing Brainwave Activity

| Visual/Colour Games | Participants Activated Brainwaves |
|---|---|
| Delta | 0% |
| Theta | 15% |
| Alpha | 55% |
| Beta-1 | 55% |
| Beta-2 | 55% |

It was seen that 7 days participation in the Visual Stimulation activity module resulted in enhancement of mostly alpha and beta waves in the participants' frontal and partial lobes. The activation of the temporal lobe was significantly reduced, and the results indicate that new memories were encoded in this brain region and then consolidated in the frontal lobes for long-term storage. On Day 7, frontal lobe activation was dominant in more than 90% of participants, confirming its importance in storing memories. Nearly 50% of participants also showed an activation in their parietal lobes, confirming parietal lobe activation during episodic retrieval tasks.

The present results indicate that parietal lobe activation occurs in tasks that involve remembering colours (especially colours difficult to identify due to different shades of colours). The activation of the parietal lobe indicates intact decision making (retrieval of memory for colours) by most participants. Most of the participants showed increased activation of alpha, beta-1 and beta-2 brainwaves. A paired t-test calculation was carried out, the results of which are set out in Table 4 below. This Table shows that the t-test results confirm a statically significant activation of all the three brainwaves, with alpha and beta-1 being dominant in most participants.

TABLE 4
Paired t-test results for Visual Stimulation
Paired t-Test

| | | Statistically | | |
|---|---|---|---|---|
| Visual | | Significant | Size Effect (% | |
| Game | p-value | (p > 0.05) | of participants) | |
| Alpha | 0.035 | Yes | 99% | Large |
| Beta-1 | 0.037 | Yes | 97% | Large |
| Beta-2 | 0.009 | Yes | 62% | Medium |

Meditation

Participants were delivered three activities, as follows:

1. Focus Point—Participants were asked to focus on a dot and reconstruct the dot, with eyes closed.

2. Breathing—Participants were delivered guided breathing exercises.

3. Music—Participants were delivered calming music.

In this activity module, alpha wave activation (which represents relaxation) was dominant during the breathing, music, calm and hypnotic meditation sessions, with a prominent parietal lobe activation. Movement from beta activation to alpha activation in this module indicates relaxation and free from stress & anxiety.

Brain lobes other than parietal were also activated, with different types of meditation. For example, focus meditation was shown to also activate the occipital lobe; and the frontal lobe is activated with calm meditation.

Only alpha wave activation (representing calmness and relaxation) was achieved with the breathing exercises. The other activities produced dominant beta waves (representing the wandering mind). Such results appear to be appropriate in consideration of the brief timescale of the activities, but longer timescales would be expected to result in more profound alpha wave activation, and hence relaxation.

Parietal lobe activation was predominant.

The parietal lobe is involved in integrating sensory information (including pain), processing language, and creating our sense of self. It is through the parietal lobe that we know where we are in space and time. Advanced meditators have greater parietal activity when not meditating, leading to a stronger sense of self. The parietal lobe is also associated with increased alertness and empathy. Also, the precuneus, a tiny structure is hidden in the folds of the parietal lobe plays a central role in self-reflection and is known to be stimulated by breathing. All our participant, being non-practitionexs of meditation, showed an activation of parietal instead of the frontal lobe, an area that remains devoid of AD hallmarks until late in the course of the disease. This, therefore, makes this module easily applicable to people with MCI and early dementia.

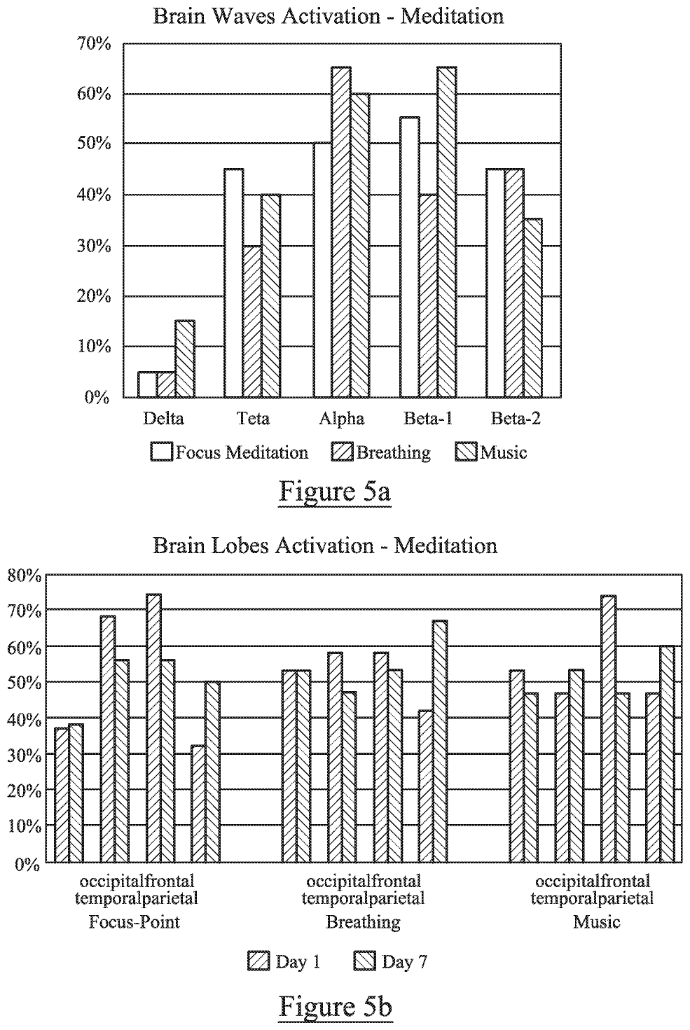

The results are presented in Table 5 and FIGS. 5a and 5b.

TABLE 5
Meditation-Percentage of Subjects showing Brainwave Activity

| | Focus Meditation | Breathing | Music |
|---|---|---|---|
| Delta | 5% | 5% | 15% |
| Theta | 45% | 30% | 40% |
| Alpha | 50% | 65% | 60% |
| Beta-1 | 55% | 40% | 65% |
| Beta-2 | 45% | 45% | 35% |

A paired t-test calculation was carried out, the results of which are set out in Table 6 below.

TABLE 6
Paired t-test results for Meditation
Paired T-Test Calculation

| | | | Statistically | | |
|---|---|---|---|---|---|
| Mediation | | | Significant | Size Effect (% | |
| module | Brainwave | p-value | (p > 0.05) | of participants) | |
| Focus | Beta-1 | 0.02 | Yes | 57% | Medium |
| point | | | | | |
| Breathing | Alpha | 0.002 | Yes | 77% | Large |
| Music | Alpha | 0.08 | No | 41% | Low |
| | Beta-1 | 0.002 | Yes | 59% | Medium |

The system of the present invention may incorporate a ‘scoring’ system, in which the user is given a score after participation in a particular activity. This may encourage, and make a positive psychological impact, on the user. The score may be produced by an algorithm which will assess, for example, the time taken for successive attempts at a game; the time taken for the user to successfully complete a game; and the number of attempts need to achieve success in a game. The scoring system may be based on the average results of healthy volunteers in a particular activity.

Aspects and embodiments of the invention may be described in the following clauses:

1. A system for monitoring brain wave activity in a subject and comparing a measured brain wave activity with a reference brain wave activity pattern, said system comprising: an activator configured to stimulate brain wave activity in a subject;

one or more sensors configured to detect said stimulated brain wave activity;

a recorder configured to capture one or more property of said detected brain wave activity; and

a processor configured to compare said captured brain wave activity with one or more reference pattern(s) of brain wave activity and quantify differences between said pattern of captured brain wave activity and said one or more reference pattern(s) of brain wave activity.

2. A system as described in clause 1, wherein the activator comprises software for a number of software-driven activities.

3. A system as described in clause 1 or clause 2, wherein each sensor comprises an electrode.

4. A system as described in any one of clauses 1 to 3, wherein the one or more sensor and the recorder are provided in a wearable headset.

5. A system as described in any one of clauses 1 to 4, wherein the processor comprises an receiver with an algorithmic+system containing algorithms for analysing information relayed by the recorder and for instructing the activator.

6. A method of monitoring brain wave activity in a subject and comparing a measured brain wave activity with a reference brain wave activity pattern, which method comprises:

delivering a software driven activity programme to a subject, by means of an activator;

detecting one or more property of the brain wave activity in an area of a subject's brain under a sensor placed on said subject's head, by means of said sensor;

capturing said one of more property of brain wave activity, by means of a recorder; and

generating a pattern of said one or more property of brain wave activity and a comparison of said pattern with a reference pattern of brain wave activity, by means of a processor; said activator, sensor, recorder and processor being as described in any one of clauses 1-5.

7. A method as described in clause 6, wherein said software driven activity programme comprises games within activity modules and wherein the games may be provided at different levels of difficulty.

8. A method as described in clause 6 or clause 7, wherein said software driven activity programme is delivered for an initial period of four weeks, during which time the frequency of monitored brain waves (f_{M}) is compared with the frequency of the brain waves in said reference pattern of brain wave activity (f_{R}).

9. A method as described in clause 8, wherein, if at the end of said initial period, if no Game within an Activity Module meets a condition 8 Hz≤f_{M}≤20 Hz, the Activity Module is discontinued.

10. A method as described in clause 8, wherein an ‘Improvement Effect’ (I), defined by the difference between f_{M} and f_{R}, is analysed by the processor.

11. A method as described in clause 10, wherein the difference between f_{M} and f_{R} is designated as ‘x’, with a positive or negative value for x indicating a difference between f_{M} and f_{R}.

12. A method as described in clause 11, wherein if I=±x within the initial period, a game is continued at the same level of activity and if I=0, a game is continued at an increased level of difficulty.

13. A method as described in clause 8, wherein said software driven activity programme is delivered for a further period of four weeks, during which time the frequency of monitored brain waves (f_{M}) is compared with the frequency of the brain waves in said reference pattern of brain wave activity (f_{R}); and an ‘Improvement Effect’ (I), defined by the difference between f_{M} and f_{R} is analysed by the processor.

14. A method as described in clause 13, wherein if I=0 or I=±x, a game is continued at an increased level of difficulty.

15. A method as described in clause 13, wherein said software driven activity programme is delivered for a further period extending beyond eight weeks, during which time the frequency of monitored brain waves (f_{M}) is compared with the frequency of the brain waves in said reference pattern of brain wave activity (f_{R}); and an ‘Improvement Effect’ (I), defined by the difference between f_{M} and f_{R} is analysed by the processor.

16. A method as described in clause 15, wherein if I=±x, a game is continued at an increased level of difficulty; and if I=0, a different game is applied.

17. A method as described in any one of the clauses 6-16, amplitude and morphology of monitored brain waves is analysed by the processor.

18. A method of supporting a diagnosis of early onset dementia in a subject, which method comprises:

detecting by means of a sensor and capturing by means of a recorder, one or more property of the brain wave activity in an area of a subject's brain under said sensor, placed on said subject's head;

generating a pattern of said one or more property and comparing said pattern with a reference pattern, by means of a processor; and

determining and quantifying by means of said processor areas of difference between said generated and reference patterns of said one or more property of said brain wave activity; said activator, sensor, recorder and processor being as described in any one of clauses 1-5.

19. A method of treating early onset symptoms of dementia in a subject, which method comprises:

detecting by means of a sensor and capturing by means of a recorder, one or more property of the brain wave activity in an area of a subject's brain under said sensor, placed on said subject's head;

generating a pattern of said one or more property and comparing said pattern with a reference pattern, by means of a processor; determining and quantifying, by means of said processor, areas of difference between said generated and reference patterns of said one or more property of said brain wave activity; and delivering, by means of an activator, a software-driven activity programme to restore, maintain or enhance one or more property of brain wave activity in the brain of said subject; said sensor, recorder, processor and activator being as described in any one of clauses 1-5.

1-28. (canceled)

29. A system for monitoring cognitive function in a user, said system comprising:
an EEG headset comprising electrodes positioned in areas corresponding to a user's brain lobes, a recorder, and a transmitter;
a personal computer configured with interactive activities; and
a cloud server system comprising a private cloud platform;
characterised in that:
the private cloud platform comprises an area in which photographs and images personal to the user may be uploaded;
the private cloud platform is configured with reference EEG profiles of healthy individuals; and
the private cloud platform is configured with algorithms for
(i) analysing EEG output of the user engaged with a specific interactive activity; and
(ii) instructing the personal computer in the presentation of specific interactive activities to the user.

30. The system as claimed in claim 29, wherein said EEG profiles of healthy individuals are activity-specific, i.e., the private cloud platform is configured with an EEG profile of healthy individuals for each of said specific interactive activities.

31. The system as claimed in claim 29, wherein the personal computer is a tablet device.

32. The system as claimed in claim 29, wherein each individual activity has a series of activity levels.

33. The system as claimed in claim 29, wherein the EEG output includes identification of brain area(s) activated with a particular activity and identification of the brain wave(s) activated with a particular activity.

34. The system as claimed in claim 29, wherein the algorithm for analysing EEG output of the user engaged with a specific interactive activity includes processes for the comparison of specific properties of the brain activity of the user, in relation to said specific interactive activity, with the reference EEG profile of healthy individuals in relation to the same specific interactive activity; and for reaching a decision in respect of one of the specific interactive activity and the activity level, for instruction to the personal computer.

35. The system as claimed in claim 29, wherein the private cloud platform is configured with EEG artefacts specific to the user.

36. The system as claimed in claim 29, including means for producing a diagnostic report in respect of a user.

37. The system as claimed in claim 29, wherein avatars based on the photographs and images personal to the user are employed.

38. The system as claimed in claim 29, including at least one of:
(a) a module comprising activities which present to the user personal photographs or other personal items, with questions relating to said personal photographs or other personal items (‘Reminiscence’);
(b) a module comprising activities which present to the user puzzles relating to colours and shapes (‘Visual Stimulation’); and
(c) a module comprising activities which present to the user focus, breathing, meditation and/or hypnotic meditation exercises (‘Meditation’).

39. The system as claimed in claim 29, including a scoring system, in which the user is given a score after participation in a particular activity, which score is produced by an algorithm which assesses at least one of: the time taken for successive attempts at a game; the time taken for the user to successfully complete a game; and the number of attempts need to achieve success in a game, said scoring system being based on the average results of healthy volunteers for a particular activity.

40. The system as claimed in claim 29, wherein, in use,
(i) electrodes in the EEG headcap monitor the brain activity of a user, in repeated sessions over a fixed time period during which time a given activity is continued without change;
(ii) algorithms in the cloud server analyse the user's brain activity over the fixed period with reference to stored reference profiles of the brain activity of healthy individuals and identify differences between the user's response and that of the healthy individuals;
(iii) at the end of the fixed period, algorithms in the cloud server direct the presentation (via the personal computer) of specific activities or levels of specific activities to the user (via the personal computer), which specific activities or levels of specific activities are directed at reducing differences in the users brain activity and said reference profiles; and
(iv) steps (i) to (iii) are repeated until the EEG output of the user matches the stored reference profiles of the brain activity of healthy individuals, for all given activities.

41. The system as claimed in claim 40, configured to analyse at least one of the frequency, amplitude and morphology of monitored brain waves.

42. The method of analysing brain activity in a patient, which method comprises:
delivering an interactive activity programme to a subject, by means of a personal computer;
detecting at least one property of the brain activity in an area of a subject's brain under a sensor placed on said subject's head, by means of said sensor;
capturing said at least one property of brain activity, by means of a recorder; and
analysing said at least one property of brain activity by means of algorithms which quantify properties of said at least one property of brain activity.

43. The method as claimed in claim 42, wherein said interactive activity programme comprises games within activity modules and wherein the games may be provided at different levels of difficulty.

44. The method as claimed in claim 42, wherein said interactive activity programme is delivered for an initial period of four weeks, during which time the frequency of monitored brain waves (f_{M}) is compared with the frequency of the brain waves in said reference pattern of brain wave activity (f_{R}).

45. The method as claimed in claim 42, wherein said interactive activity programme is delivered for a further period of four weeks, during which time the frequency of monitored brain waves (f_{M}) is compared with the frequency of the brain waves in said reference pattern of brain wave activity (f_{R}); and an ‘Improvement Effect’ (I), defined by the difference between f_{M} and f_{R} is analysed.

46. The method as claimed in claim 45, wherein if I=0 or I=±x, a game is continued at an increased level of difficulty.

47. The method as claimed in claim 42, wherein at least one of the frequency, amplitude and morphology of monitored brain waves is analysed.

48. A method for improving cognition in a patient, the method comprising providing to the patient the system of claim 29 for a fixed period of time.
